(19) United States
(12) Patent Application Publication
(10) Pub. No.: US 2007/0190114 A1
(43) Pub. Date: Aug. 16, 2007

(54) Antioxidant health lozenges

(76) Inventor: William Joseph Smart, Castle Rock, CO (US)

Correspondence Address: BELL, BOYD & LLOYD, LLP, P.O. Box 1135, CHICAGO, IL, 60690, US

(21) Appl. No.: 11/351,864

(22) Filed: Feb. 10, 2006

(51) Int. Cl. A61K 36/82; A61K 36/53; A61K 38/05; A61K 38/43; A61K 31/385; A61K 31/355; A61K 31/353; A61K 9/68

(52) U.S. Cl. 424/440; 424/729; 424/745; 514/18; 514/456; 514/458; 514/474; 424/702; 424/94.1; 514/440

(57) ABSTRACT

Compositions and methods related to combining antioxidant-promoting agents and breath masking agents in a single oral delivering formulation are provided. The composition includes a blend of antioxidants directed to reducing oxidative stress associated with smoking and exposure to other environmental toxins. The active ingredients of the oral composition are in a form that remains unaltered as the active ingredients are rapidly absorbed through the oral mucosa directly into systemic circulation.

### BACKGROUND

The present disclosure describes compositions and methods related to health products that combine a breath mint with a blend of antioxidant supplements that enhance the internal defenses of an individual to reduce oxidative stress caused by tobacco smoke.

Excessive production of reactive oxygen species known as free radicals in the presence of diminished antioxidant substances can lead to oxidative stress. Free radicals are the natural by-product of oxidative phosphorylation during cellular respiration occurring in the mitochondria of cells in the body to make the high-energy molecule, adenosine triphosphate (ATP). Electrons unable to be utilized during aerobic respiration can combine with oxygen to form a highly reactive oxidative species having an unpaired single electron such as superoxide, peroxide, hydroxyl radical, and other free radicals derived from oxygen. In the absence of sufficient levels of antioxidants, a free radical chain reaction can propagate generating endogenous oxidative stress in human cells and tissues. In particular, these highly reactive free radicals present during oxidative stress can react with essential biomolecules such as DNA and RNA, enzymes and other proteins, and membrane phospholipids to compromise their structure and eventually lead to cell death.

The body's defense against oxidative stress is accomplished by interconnecting systems of non-enzymatic micronutrients antioxidants, such as glutathione and coenzyme Q, along with enzymatic processes. Antioxidants are responsible for metabolizing free radicals in order to maintain a balance between metabolic reactions such as metabolic activation and detoxification, formation and scavenging of free radicals and DNA damage and repair.

A major antioxidant produced by the cell which is critically important for protecting the cell from free radicals is a tripeptide known as glutathione. Glutathione is a small molecule found in almost every cell. Glutathione is involved in both reduction reactions to quench free radicals and conjugation reactions to form a complex that can be removed by the body. As an antioxidant, the reduced form of glutathione is able to react with the single electron of free radical species or accept lone pairs of electrons, thereby neutralizing the free radical and short-circuiting any free-radical chain reaction. In addition to its role as an antioxidant, glutathione also recycles Vitamin E and other well-known antioxidants such as Vitamin C, keeping them in their active state. Therefore, glutathione plays a role in the final trapping of free radicals and is necessary to maintain a normal balance between oxidation and anti-oxidation.

For example, reduced glutathione (GSH) can neutralize a hydroxyl radical (.OH):
GSH+^{•}OH→^{•}GS+H₂O
Oxidized glutathione radicals (^{•}GH) can then neutralize each other to form oxidized glutathione (GSSG):
^{•}GH+^{•}GH→GSSG
Glutathione reductase then adds hydrogens to the oxidized glutathione to regenerate reduced glutathione (GSH).

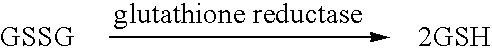

When an individual is exposed to sources of free radicals from carcinogens and other environmental toxins, in addition to endogenous sources of free radical production, the balance between defensive metabolic reactions may be upset. Accordingly, the excessive production of reactive oxygen species associated with oxidative stress can saturate detoxification mechanisms. If the detoxification mechanisms are saturated and overwhelmed, carcinogenic compounds are not disabled and are left to damage cells and tissues leading to tumor growth and other diseases.

The pre-carcinogens and free radicals formed from tobacco smoke cigarettes or other forms of burning tobacco taken in through the mouth can produce oxidative stress. Every puff of smoke delivers over 10¹⁴ oxidizing molecules that must be chemically reduced by cellular mechanisms using antioxidants to quench the free radicals formed. Thus, the free radicals produced from smoking can rapidly deplete plasma and tissue levels of antioxidants, producing an enormous oxygen free radical load. The resulting demand on tissue antioxidant systems leads to severe antioxidant deficiency states and contributes to the pathogenesis of disease related to smoking.

Depletion of plasma and tissue levels of antioxidants by oxygen free radicals also contributes to inflammation and affects the immune system as indicated by increased plasma levels of inflammatory cytokines derived from alveolar macrophages in response to oxidative stress. Inflammation of the alveolae can subsequently lead to chronic obstructive pulmonary disease. Smoking is also among the major coronary risk factors. Cigarette smoking, in particular, causes harmful effects to occur on the heart and blood vessels as a result of either active smoking or passive smoking. The relationship between cigarette smoking and cardiovascular disease is independent of other coronary risk factors. For example, smoking increases the risk of developing peripheral artery disease, coronary artery disease and cerebrovascular disease. Chemicals released from the tobacco, such as nicotine, are toxic to vascular endothelium. Cigarette smoking has also been shown to increase low-density lipoprotein cholesterol levels and decreases high-density lipoprotein cholesterol levels associated with atherosclerosis in peripheral vascular disease. Merck Index, §16, page 1656.

Smoking has also been shown to damage the DNA of cells leading to cell death or tumorigenesis. One of the biomarkers indicating damage to the DNA of cells caused by smoking, and cigarette smoking in particular, is sister chromatid exchange frequency. During mitosis, a cell replicates its DNA generating two complete sets of its genome before dividing. For example, the DNA of the forty-six chromosomes in a human cell is replicated to generate two complete sets of forty-six chromosomes, for a total of ninety-two chromosomes. Each original chromosome and its identical copy of homologous stretches of DNA sequence are known as sister chromatids. During the DNA replication phase of mitosis, sister chromatids pair up and homologous stretches of DNA sequence are exchanged between the sister chromatids. However, when the DNA of a cell is damaged by genotoxic agents, such as free radicals, the rate of sister chromatid exchange increases as a means of repairing the DNA damage caused by the genotoxic agents. Sustained levels of high frequency sister chromatid exchange can lead to cell death when these repair mechanisms shut down. Therefore, exposure to free radicals generated from smoking increases the frequency in which sister chromatid exchange occurs and leads to the eventual death of cells affected by oxidative stress.

In an effort to supply antioxidants to the body, antioxidants have been included in a variety of nutritional supplements. Many of these supplements include the use of high-dose single antioxidants. This poses potential risks by perturbing the antioxidant-oxidant balance discussed above. In other words, supplying only a single antioxidant in a system that involves multiple metabolic mechanisms will not reduce oxidative stress if other systems are overloaded. Therefore, the overall ability to successfully trap free radicals will be ineffective, thus leading to further oxidative stress.

In addition, glutathione, a tripeptide, may be digested with proteases during digestion and may not enter mucosal cells intact. In fact, because glutathione is a peptide that would not escape digestion, there would be little, if any, intact glutathione when ingested as such. Consequently, glutathione must be synthesized inside the cell from its three constituent amino acids: glycine, glutamate and cysteine. The rate at which glutathione can be made depends on the availability of cysteine.

Although there are about one billion smokers world-wide and nearly fifty million smokers just in the United States, there are virtually no products that address the health needs of smokers and of people exposed to second hand smoke and other environmental toxins and carcinogens. Most products available for smokers are directed to smoking cessation. However, most smokers are either not trying to quit smoking or have been unsuccessful in quitting. In fact, only one out of forty smokers who attempt to quit smoking are successful. In addition, there are few, if any, breath freshening products that specifically target the specific needs of smokers. Until a smoker is able to quit, there is, therefore, a need for a product that is designed to efficiently deliver an effective combination of antioxidants to mitigate at least some of the long term health risks associated with smoking, to resolve some of the damage elicited by oxidants present in smoke and to reduce oxidative stress.

### SUMMARY

The present disclosure describes compositions and methods related to a health product that combines a breath mint with a blend of antioxidant supplements that enhance the internal defenses of an individual to reduce oxidative stress caused by tobacco smoke. In particular, the health product is directed to mitigating the effects of the toxins and carcinogens particularly associated with smoking. Certain components of the health product are designed to be retained in the mouth to freshen breath while other components are readily absorbed through the oral/buccal mucosa to rapidly deliver sustained amounts of antioxidants.

In an embodiment, a lozenge is provided for preventing or reducing oxidative stress. The lozenge comprises an effective amount of at least one precursor of glutathione.

In an embodiment, the precursor of glutathione is in a form suitable to be substantially absorbed through an oral mucosa of an individual.

In an embodiment, the precursor of glutathione production is selected from the group consisting of N-acetyl cysteine, alpha-lipoic acid, selenium and combinations thereof.

In an embodiment, the lozenge includes at least one of the compounds selected from the group consisting of ascorbic acid, a tocopherol, a polyphenol, a flavonoid, a quinone and combinations thereof.

In an embodiment, the tocopherol includes d-alpha tocopherol.

In an embodiment, the polyphenol is derived from a plant extract selected from the group consisting of a green tea extract, rosemary extract and combinations thereof.

In an embodiment, the polyphenol is chosen from the group consisting of resveratrol, carnosol, catechin, gallaogatechin, epicatechin, epigallocatechin, epicatechin gallate, epigallocatechin gallate and combinations thereof.

In an embodiment, the flavonoid includes quercetin.

In an embodiment, the quinone includes coenzyme Q.

In an embodiment, the lozenge includes at least one agent for reducing halitosis.

In another embodiment, a method of preventing or reducing oxidative stress in an individual exposed to tobacco smoke is provided. The method includes administering to the individual a lozenge comprising an effective amount of at least one precursor of glutathione.

It is therefore an advantage of the compositions and methods disclosed herein to provide an oral composition having a blend of antioxidants specifically combined to treat oxidative stress caused by smoking.

Another advantage of the compositions and methods disclosed herein includes providing a composition formulated to deliver an effective amount of antioxidants across the oral mucosa of the mouth of a user.

Yet another advantage of the compositions and methods disclosed herein includes administering a composition that is rapidly absorbed into systemic circulation without altering the ingredients of the composition.

Another advantage of the compositions and methods disclosed herein includes providing a composition formulated to undergo sublingual absorption to provide a faster means of replenishing needed antioxidants in the blood stream.

A further advantage of the compositions and methods disclosed herein includes providing a composition capable of delivering an effective amount of at least one stable substance that promotes the production of an efficacious antioxidant, such as glutathione.

An additional advantage of the compositions and methods disclosed herein includes providing an oral substance that can be manipulated in the mouth to contribute to satisfying the “oral fixation” component of a smoking habit, thereby replacing the need for a cigarette as an “oral fixation” substitute, particularly during periods when the smoker is not able to smoke.

An additional advantage of the compositions and methods disclosed herein includes combining substances that mask bad breath and that reduce oxidative stress in a single formulation.

Another advantage of the compositions and methods disclosed herein includes providing a form of antioxidant intake that fits the lifestyle of smokers in that a majority of smokers use mints already; thus, adding needed nutrients to a breath mint does not require a behavorial change in the smoker.

An advantage of the compositions and methods disclosed herein includes repeated or continuous dosing of nutritional bioactives throughout the day to sustain higher plasma concentration levels of key antioxidant molecules.

Additional features and advantages are described herein, and will be apparent from, the following Detailed Description and the figures.

### DETAILED DESCRIPTION

The present disclosure describes compositions and methods related to a health product that combines a breath mint with a blend of antioxidant supplements into a single formulation. The antioxidants of the health product enhance the internal defenses of an individual to reduce oxidative stress caused, at least in part, by exposure to tobacco smoke. In particular, the health product can be directed to mitigating the effects of the toxins and carcinogens particularly associated with smoking. Certain components of the health product can be designed to be retained in the mouth to freshen breath while other components can be specifically designed to be readily absorbed through the oral mucosa to rapidly deliver amounts of antioxidants that each or collectively constitute an effective amount of antioxidant.

The antioxidant-rich cocktail composition addresses a smokers' need for fresh breath while providing a product that will also boost the deficient antioxidant status known to exist in people who habitually smoke cigarettes. Combining a breath mint with a blend of antioxidant supplements can boost a smoker's defenses against illness associated with oxidative stress such as atherosclerosis, heart and lung disease, and many types of cancers.

In an embodiment, specific dosages of micronutrient antioxidant molecules are administered to an individual in need thereof. Although single antioxidant micronutrients have been tested in human trials and have been shown to be somewhat effective in reducing risk of certain diseases, in an embodiment, antioxidants are administered in combination rather than as single supplements. There are many similar biochemical processes that oxidize reduced antioxidant molecules to neutralize free radicals and then restore the antioxidant molecules to a reduced state. Optimal concentrations of each of the micronutrient antioxidants can be administered in order to promote this synergistic interaction between the antioxidants to recycle antioxidants and to form a biochemical chain of defense against free radicals associated with environmental toxins such as tobacco smoke.

In an embodiment, most of the micronutrient antioxidant molecules included in the composition can be rapidly absorbed into the systemic circulation of the user via buccal or sublingual absorption across the oral mucosa. In addition, the antioxidant compounds absorbed sublingually will bypass the digestion process prior to absorption through enterocytes in the gut.

As described herein, glutathione is naturally produced in the body but is ineffective when ingested and metabolized in the gastrointestinal tract. Therefore, in an embodiment, the composition includes precursors or substances that promote the production of glutathione or enhance its antioxidant activity. In an embodiment, the composition includes at least one of the precursors or substances that contribute to the production or action of glutathione selected from the group consisting of N-acetyl cysteine, α-lipoic acid and selenium.

As discussed above, the rate at which glutathione can be made depends on the availability of cysteine. However, cysteine itself is toxic and can be made to be ineffective during digestion unless chemically modified. By providing an acetylated cysteine in the form of, for example, N-acetyl cysteine, the cysteine becomes more stable until it is needed to produce glutathione and is rapidly absorbed into cells and tissues. N-acetyl cysteine is readily de-acetylated in the presence of oxidative stress when increased levels of glutathione are needed. Additionally, N-acetyl cysteine is, by itself, an effective cancer chemopreventive agent at a daily dose of 600 mg. [Cancer Epidemiol Biomarkers Prev. 2002 February; 11(2):167-75; Am J Physiol Lung Cell Mol Physiol. 2002 August; 283(2):L409-17.] Therefore, N-acetyl cysteine can be provided in a suitable dosage form of the composition to allow between about 2 μg and about 3000 mg to be administered to an individual per day.

Lipoic acid induces cystine/cysteine uptake, thereby increasing synthesis of glutathione. Along with increasing production of glutathione, α-lipoic acid also acts to balance vitamin C and E antioxidant levels. Alpha lipoic acid is the synthetic form of lipoic acid and is a racemic mixture of the R-form and S-form of enantiomers. Although the R-enantiomer is more biologically active than the S-enantiomer, each enantiomer exerts a synergistic effect on the reduction of the other enantiomer to result in greater formation of its reduced form, dihydrolipoic acid. Accordingly, alpha lipoic acid is readily absorbed into most tissues and is rapidly converted to its reduced form by NADH or NADPH. Both the oxidized and reduced forms of lipoic acid are anti-oxidants. By donating two hydrogens, the reduced form of lipoic acid can neutralize free radicals without itself becoming a free radical and can regenerate vitamin C, vitamin E, coenzyme Q-10, and L-glutathione from their oxidized forms. A synergistic effect is therefore believed to occur through the interaction of alpha-lipoic acid and other antioxidants. Therefore, alpha lipoic acid can be provided in a suitable dosage form of the composition to allow between about 1 μg and about 1000 mg to be administered to an individual per day.

Selenium is an essential component or co-factor of the enzyme glutathione peroxidase, which uses glutathione to neutralize hydrogen peroxide to terminate the free radical cascade. The enzyme glutathione peroxidase utilizes reduced glutathione to neutralize the oxidative effects of hydrogen peroxide:
2GSH+H₂O₂-->GSSG+2H₂O

Selenium deficiencies result in glutathione peroxidase depletion. Selenium achieves a protective effect against many cancers, but, an excess amount of selenium can be toxic. Therefore, selenium can be provided in a suitable dosage form of the composition to allow between about 1 μg and about 1000 μg to be administered to an individual per day. Selenium may be present either as a salt, e.g. sodium selenite, selenium clycinate, or as an amino acid chelate, e.g. selenomethionine.

The composition can also include a tocopherol such as alpha-tocopherol (vitamin E). Vitamin E donates a hydrogen to neutralize a free radical. The vitamin E can be provided in a suitable dosage form to allow between about 400 IU to about 3,200 IU to be administered to an individual per day. In an embodiment, vitamin E can be provided in a suitable dosage form to allow between about 1 IU to about 1500 IU to be administered to an individual per day.

In an embodiment, ascorbic acid (Vitamin C) is combined with Vitamin E in the composition to promote synergistic biochemical interactions between these two antioxidants. Vitamin C can be provided in a suitable dosage form to allow between about 500 mg to about 5,000 mg to be administered to an individual per day. Vitamin C in combination with Vitamin E significantly diminishes oxidative damage to lipids by decreasing chromosomal instability in lymphocytes. [Free Radic Res. 2005 June: 39(6):659-66.]

In an embodiment, polyphenols and flavonoids present in green tea extract can be included in the composition to reduce damage of proteins caused by smoking and other environmental toxins. In particular, the bioflavonoid epigallocatechin gallate has been shown to stabilize sister-chromatid exchange in peripheral lymphocytes in smokers to a level comparable to non-smokers suggesting a strong potential to counter adverse damage of cigarette smoke [J. Nutr. 2003 August; 133(8):2622-8; Clin Diagn Lab Immunol. 2002 July; 9(4):864-71; Cancer Epidemiol Biomarkers Prev. 1995 June; 4(4):387-91]. In addition to epigallocatechin gallate the composition can also include catechin, gallocatechin, epicatechin, epigallocatechin, epicatechin gallate, theaflavin, theaflavin gallate, theaflavin gallate, theaflavin digallate, thearubigins, proanthocyanidins and other suitable polyphenols and flavonoids. Therefore, green tea extract can be provided in a suitable dosage form of the composition to allow between about 1 μg and about 500 mg EGCG to be administered to an individual per day. It should be appreciated that the dosage may be in terms of epigallocatechin gallate or green tea extract.

Other polyphenols and flavonoids included in the composition may be found in vascular plants such as rosemary extracts as well as in many fruits, vegetables, teas, wines, nuts, seeds, roots and bee pollen. Rosemary extract, in particular, contains many antioxidants and anticarcinogens including carnosol. Carnosol shows potent antioxidative activity in free radical scavenging to reduce oxidating stress. Therefore, rosemary can be provided in a suitable dosage form of the composition to allow between about 1 μg and about 1000 mg to be administered to an individual per day.

Another polyphenolic compound which can be included in the composition includes resveratrol. Resveratrol is an antioxidant, cardioprotectant and antimutagenic agent. As an anti-inflammatory, resveratrol inhibits basal release of inflammatory cytokine from alveolar macrophages in smokers. [Thorax. 2003 November; 58(11):942-6]. Therefore, resveratrol can be provided in a suitable dosage form of the composition to allow between about 1 μg and about 1000 mg to be administered to an individual per day.

Other antioxidants included in the composition can include quinines such as coenzyme Q-10 and flavonoids such as quercetin. Coenzyme Q-10 and quercetin demonstrate effectiveness in reducing the damage from smoking-associated diseases, particularly cardiovascular diseases. Quercetin is an active flavonoid that serves as the backbone for many other flavonoids. Quercetin is best known as an anti-inflammatory agent shown to stabilize mast cell membranes affected by smoking by inhibiting inflammatory processes. Therefore, quercetin can be provided in a suitable dosage form of the composition to allow between about 1 μg and about 1000 μg to be administered to an individual per day.

Coenzyme Q-10 is a quinone compound that serves as an electron carrier between various flavoproteins and in cellular respiration. Coenzyme Q-10 is also and effective antioxidant. Specifically, coenzyme Q-10 has two hydrogens available to be donated to neutralize a free radical such as an oxidized form of Vitamin E to reduce and regenerate Vitamin E. Coenzyme Q-10 is able to donate both hydrogens to avoid becoming a free radical itself and is, therefore, capable of breaking the free radical chain reaction. Accordingly, coenzyme Q is indicated in the treatment of a wide variety of degenerative diseases including diabetes, stroke, cancer and especially heart disease. Therefore, coenzyme Q-10 can be provided in a suitable dosage form of the composition to allow between about 1 μg and about 1000 μg to be administered to an individual per day.

Accordingly, in an embodiment, the dosage of ascorbic acid included in the composition may be between about 1 mg and about 5000 mg or between about 0.1 percent and about 50 percent by weight of the composition.

In an embodiment, the dosage of d-alpha tocopheryl included in the composition may be between about 1 IU and about 1500 IU or between about 0.1 percent and about 50 percent by weight of the composition.

In an embodiment, the dosage of selenium included in the composition may be between about 1 μg and about 1000 μg or between about 0.1 percent and about 20 percent by weight of the composition.

In an embodiment, the dosage of green tea extract included in the composition may be between about 0.1 mg and about 500 mg or between about 0.1 percent and about 50 percent by weight of the composition.

In an embodiment, the dosage of N-acetyl cysteine included in the composition may be between about 1 μg and about 1000 mg or between about 0.5 percent and about 50 percent by weight of the composition.

In an embodiment, the dosage of resveratrol included in the composition may be between about 1 μg and about 1000 mg or between about 0.1 percent and about 50 percent by weight of the composition.

In an embodiment, the dosage of quercetin included in the composition may be between about 0.1 mg and about 1000 mg or between about 0.01 percent and about 10 percent by weight of the composition.

In an embodiment, the dosage of rosemary extract included in the composition may be between about 10 μg and about 100 mg or between about 0.01 percent and about 5 percent by weight of the composition.

In an embodiment, the dosage of alpha lipoic acid included in the composition may be between about 1 μg and about 100 mg or between about 0.01 percent and about 5 percent by weight of the composition.

In an embodiment, the dosage of coenzyme Q-10 included in the composition may be between about 1 μg and about 1000 mg or between about 0.01 percent and about 5 percent by weight of the composition.

It should be appreciated that any combination of the active ingredients described herein can be included in the composition. It should be further appreciated that each of the active ingredients of the oral composition described herein may be present in the composition in a range from about 0.01% to about 90% by weight of the composition.

### EXAMPLE I

One example of a formulation of the composition can include the following combination of active agents:

| | | | |
|---|---|---|---|
| Vitamin C | 120 | mg | 4.40% by weight of the composition |
| (ascorbic acid) | | | |
| Vitamin E | 65 | IU | 1.75% by weight of the composition |
| d-alpha tocopheryl | | | |
| Selenium | 30 | μg | 1.10% by weight of the composition |
| (clycinate/chelate) | | | |
| Green Tea Extract | 30 | mg | 1.10% by weight of the composition |
| 98% polyphenols | | | |
| N-Acetyl Cysteine | 75 | mg | 2.75% by weight of the composition |
| Resveratrol | 1 | mg | 0.04% by weight of the composition |
| Quercetin | 1 | mg | 0.04% by weight of the composition |
| Rosemary extract | 1 | mg | 0.04% by weight of the composition |
| Alpha Lipoic acid | 0.5 | mg | 0.02% by weight of the composition |
| Coenzyme Q-10 | 0.5 | mg | 0.02% by weight of the composition |

In an embodiment, the composition is included in a formulation adapted to mask or reduce halitosis, such as a mint, lozenge, gum, strip, or any other form of administering an effective amount of an active agent designed to reduce halitosis. To this end, the formulation can include a menthol or any other suitable physiological cooling agent as is well known in the art. Therefore, in addition to the health benefits achieved in delivering an effective amount of antioxidants, the disclosed formulations can act to mask or reduce halitosis associated with smoking and other sources of bad breath.

Other ingredients can include flavorings, sweeteners, colorings, excipients and other fillers. For example, the compositions can include any suitable flavoring, alone or in combination with any of the above. Flavorings can include, but are not limited to, spearmint, peppermint, lemon, lime, orange, cinnamon, licorice and the like, alone or in combination. In an embodiment, natural and artificial flavoring agents can be combined in any fashion acceptable to the senses.

The compositions can include any suitable bulk sweetener, alone or in combination with any of the above. Bulk sweeteners can include, but are not limited to, starch, dextrose, cellulose, maltodextrin, hydrogenated starch hydolysates and the like, alone or in combination.

The compositions can include any suitable softeners or plasticizing agents, alone or in combination with any of the above. Softeners can include, but are not limited to, glycerin, lecithin and the like, alone or in combination.

The compositions can include any suitable sweetening agents, alone or in combination with any of the above. Sweeteners can include, but are not limited to, glycerol, propylene glycol, sorbitol, sucrose, saccharinsorbitol, hydrogenated starch hydolysates, corn syrup and the like, alone or in combination.

The compositions can include any suitable sugar alcohols, alone or in combination with any of the above. Sugar alcohols can include, but are not limited to, mannitol, maltitol powders and the like, alone or in combination.

The compositions can include any suitable high-intensity artificial sweeteners, alone or in combination with any of the above. High-intensity artificial sweeteners can include, but are not limited to, sucralose, aspartame, salts of acesulfame, altitame, saccharin and its salts, cyclamic acid and its salts, glycerrhizinate, dihydrochalcones, thaumatin, monellin, and the like, alone or in combination.

The compositions can include any suitable dispersing agents, alone or in combination with any of the above. Dispersing agents can include, but are not limited to, potato starch and sodium starch glycollate and combinations thereof.

The compositions can include any suitable surface active agents, alone or in combination with any of the above. Surface active agents can include, but are not limited to, sodium lauryl sulphate and the like, alone or in combination.

The compositions can include any suitable diluents, alone or in combination with any of the above. Diluents can include, but are not limited to, calcium carbonate, sodium carbonate, lactose, microcrystalline cellulose, calcium phosphate, calcium hydrogen phosphate, sodium phosphate and the like, alone or in combination.

The compositions can include any suitable granulating and disintegrating agents, alone or in combination with any of the above. Granulating and disintegrating agents can include, but are not limited to, corn starch, alginic acid and the like, alone or in combination.

The compositions can include any suitable binding agents, alone or in combination with any of the above. Binding agents can include, but are not limited to, gelatin, acacia, pre-gelatinized maize starch, polyvinylpyrrolidone, hydroxypropyl methylcellulose and the like, alone or in combination.

The compositions can include any suitable lubricating agents, alone or in combination with any of the above. Lubricating agents can include, but are not limited to, magnesium stearate, stearic acid, silica, talc and the like, alone or in combination.

The compositions can include any suitable coloring agent, alone or in combination with any of the above.

The compositions can include any suitable low calorie bulking agent, alone or in combination with any of the above. Low caloric bulking agents can include, but are not limited to, polydextrose, raftilose, raftilin, fructooligosaccharides, palatinose oligosaccharide, guar gum hydrolysate, indigestible dextrin and the like, alone or in combination.

Compositions may be prepared, packaged, and/or sold as a buccal or sublingual formulation. Buccal absorption in addition to, or in combination with, sublingual absorption of antioxidants acts to promote rapid absorption of the composition via the blood vessels in the oral mucosa (buccal) or under the tongue (sublingual). Rapid absorption of the antioxidants of the formulation allows antioxidants to be delivered into systemic circulation at a consistent rate to maintain plasma levels of these oxygen-fighting molecules for extended periods of time depending on the rate of consumption throughout the day. Achieving a steady state of sustained levels of these nutrients/antioxidants in blood plasma as well as in the tissues will prevent the depletion of plasma and tissue levels of antioxidants enabling sufficient antioxidants to be available to quench the increased quantities of free radicals to reduce oxidative stress. Absorption of antioxidants through the gastrointestinal tract may also occur.

Accordingly, the composition may be formulated as an oral formulation made using known methods. Oral formulations may be prepared, packaged, and/or sold as a discrete solid dose unit, each containing a predetermined amount or dose of the active ingredient. The formulations are preferably in the form of a lozenge or any other form suitable for oral delivery of the disclosed compositions. As used herein, the term “lozenge” can refer to any solid or semi-solid substrate wherein at least a majority of the substrate is designed to dissolve in an oral cavity. Other forms of the composition may be prepared using known methods. Such formulations may be administered directly to a subject or used to prepare other forms of the composition. For example, in an embodiment, the composition may be administered in an effervescent form and packaged in individual packets or as tablets to be dissolved or dispersed to form a solution or suspension in water or other beverages for administration prior to oral administration.

Each formulation may contain an effective amount of the composition. As referred to herein, an effective amount includes an amount of at least one antioxidant that contributes to reducing or preventing the occurrence of oxidative stress. In addition, an effective amount of composition can include an amount of a substance that masks or reduces halitosis. The remainder of the formulation may contain any suitable physiologically acceptable ingredient as described herein or in addition to the ingredients described herein.

The compositions may be prepared, packaged, or sold as tablets. Tablets containing the composition of the present invention may be made by compressing or molding, in a suitable device, the active ingredient(s) alone or together with one or more additional ingredients. Compressed tablets may be prepared by compressing the active ingredient, when it is in a free-flowing form such as a powder or granular preparation, alone or together with one or more binders, lubricants, excipients, surface active agents, or dispersing agents as described above. Molded tablets may be made by molding a mixture of the active ingredient, a pharmaceutically acceptable carrier, and sufficient liquid to moisten the mixture. Pharmaceutically acceptable excipients used for manufacturing tablets include inert diluents, granulating and disintegrating agents, binding agents, and lubricating agents as described above.

Controlled- or sustained-release formulations of the composition may be produced using conventional technology. To this end, the active agents may be adapted as is known in the art to achieve delayed release or distribution of the active agents of the composition to an individual. Coating a lozenge, for example, may provide sustained release of the active ingredient from within the coated portion of the formulation. Glyceryl monostearate, glyceryl distearate or any other suitable coating may be used to coat the oral formulations. Additionally or alternatively, the active agents in a solid or semi-solid oral formulation may be associated with particular layers, coatings or any other portion of the composition to effect a desired rate of release of the active agents from the composition.

In an embodiment, the compositions described above can be administered to an individual exposed to, or at risk of exposure to, toxins, carcinogens or any other pharmacologically active, antigenic, cytotoxic, mutagenic or carcinogenic substance, particularly associated with smoking. As referred to herein, exposure can be direct, as with one who smokes a cigarette, or indirect, as with one who inhales smoke from another source, such as second-hand smoke.

Methods include administering an effective amount of at least one antioxidant to an individual in need thereof to reduce oxidative stress associated with exposure to toxins and carcinogens particularly associated with smoking. Methods can also include providing a health product comprising at least one component designed to freshen breath and at least one component designed to be readily absorbed through the oral mucosa to rapidly deliver an effective amount of at least one antioxidant.

The health product can be administered as needed, throughout the day, after a smoking session, such as after smoking at least one cigarette, or any other suitable frequency and/or period of time.

It should be understood that various changes and modifications to the embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A lozenge for preventing or reducing oxidative stress comprising an effective amount of at least one precursor of glutathione.

2. The lozenge of claim 1, wherein the precursor of glutathione is in a form suitable to be substantially absorbed through an oral mucosa of an individual.

3. The lozenge of claim 1, wherein the precursor of glutathione is selected from the group consisting of N-acetyl cysteine, alpha-lipoic acid, selenium and combinations thereof.

4. The lozenge of claim 1, which includes at least one of the compounds selected from the group consisting of ascorbic acid, a tocopherol, a polyphenol, a flavonoid, a quinone and combinations thereof.

5. The lozenge of claim 4, wherein the tocopherol includes d-alpha tocopherol.

6. The lozenge of claim 4, wherein the polyphenol is derived from a plant extract selected from the group consisting of a green tea extract, rosemary extract and combinations thereof.

7. The lozenge of claim 6, wherein the polyphenol is chosen from the group consisting of resveratrol, carnosol, catechin, gallaogatechin, epicatechin, epigallocatechin, epicatechin gallate, epigallocatechin gallate and combinations thereof.

8. The lozenge of claim 4, wherein the flavonoid includes quercetin.

9. The lozenge of claim 4, wherein the quinone includes coenzyme Q-10.

10. The lozenge of claim 1, which includes at least one agent for reducing halitosis.

11. A method of preventing or reducing oxidative stress in an individual exposed to tobacco smoke comprising administering to said individual a lozenge comprising an effective amount of at least one precursor of glutathione.

12. The method of claim 11, wherein the precursor of glutathione is in a form suitable to be substantially absorbed through the oral mucosa of the individual.

13. The method of claim 11, wherein the precursor of glutathione is selected from the group consisting of N-acetyl cysteine, alpha-lipoic acid, selenium and combinations thereof.

14. The method of claim 11, which includes at least one of the compounds selected from the group consisting of ascorbic acid, a tocopherol, a polyphenol, a flavonoid, a quinone and combinations thereof.

15. The method of claim 14, wherein the tocopherol includes d-alpha tocopherol.

16. The method of claim 14, wherein the polyphenol is derived from a plant extract selected from the group consisting of a green tea extract, rosemary extract and combinations thereof.

17. The method of claim 16, wherein the polyphenol is chosen from the group consisting of resveratrol, carnosol, catechin, gallaogatechin, epicatechin, epigallocatechin, epicatechin gallate, epigallocatechin gallate and combinations thereof.

18. The method of claim 14, wherein the flavonoid includes quercetin.

19. The method of claim 14, wherein the quinone includes coenzyme Q-10.

20. The method of claim 11, wherein the lozenge includes at least one agent for reducing halitosis.
